(19) United States
(12) Patent Application Publication
(10) Pub. No.: US 2023/0178956 A1
(43) Pub. Date: Jun. 8, 2023

(54) METHOD FOR OPERATING DIODE-PUMPED PULSED LASERS

(71) Applicant: DORNIER MEDTECH LASER GMBH, Wessling (DE)

(72) Inventor: Werner HIERETH, Birgland (DE)

(21) Appl. No.: 17/997,989

(22) PCT Filed: May 5, 2021
(86) PCT No.: PCT/EP2021/061770

§ 371 Date: Nov. 4, 2022

(30) Foreign Application Priority Data
May 5, 2020 (EP) 20172968.8

(51) Int. Cl. H01S 3/094; H01S 3/0941; H01S 3/16; H01S 3/091

(52) U.S. Cl. CPC H01S 3/094076; H01S 3/0941; H01S 3/1616; H01S 3/1643; H01S 3/0912; H01S 5/4025

(57) ABSTRACT

The present invention relates to a method for operating a pulsed diode-pumped solid-state laser comprising: providing a pump light source for pumping a solid-state laser, said pump light source comprising at least one laser diode unit configured for emitting a series of light pulses for pumping the solid-state laser, modulating the series of light emission pulses of the at least one laser diode unit such that only the light pulses with a frequency close to or equal to a requested frequency setting of the solid-state laser are operated with a/the required pulse amplitude and/or a/the required pulse duration to trigger light emission of the solid-state laser, and such that any other light pulses of the at least one laser diode unit are operated to not trigger light emission of the solid-state laser.

### TECHNICAL FIELD

The invention relates to a method and a laser system.

### BACKGROUND AND SUMMARY

Solid-state lasers are commonly pumped using gas lamps or laser diodes as pump light sources. While gas lamps are cheaper than laser diodes, they have a shorter lifetime and further can only provide a broad spectral emission, resulting in a rather low efficiency of the pumping of solid state lasers.

Diode lasers on the other hand are much more expensive than gas lamps but have a much higher lifetime and a small spectral width (e.g. 2-3 nm) of their emission spectrum and are therefore much more efficient in the pumping of solid state lasers having spectral absorption bands with narrow widths (e.g. a few nm). However, in order to efficiently pump solid state lasers with narrow width absorption bands, diode lasers must be operated in a rather small temperature range because the emitted wavelength/emitted spectrum of the diode laser is dependent on the temperature of the laser diode and, for example, the emitted wavelength or emission spectrum peak wavelength can change or shift, for example, by ˜0.28 nm/° C.

Hence, laser diodes are designed for emitting a defined wavelength/defined emission spectrum peak wavelength only at a defined operating temperature.

While for continuous wave diode-pumped solid-state lasers (CWDPSSL) this is not a problem, since the laser diodes have a constant heat dissipation during operation, for pulsed diode-pumped solid-state lasers (PDPSSL), the temperature-wavelength relationship between the emitted wavelength of the laser diode and its temperature can inter alia depend on the duty cycle of operation and cooling parameters/operation of the laser diode.

Current techniques for pumping pulsed solid-state lasers are cumbersome, slow and not efficient, resulting, for example, in undesired reductions of the laser power output efficiency of the solid-state lasers over a broad range of desired operating frequencies of the solid-state laser.

Furthermore, current techniques for pumping pulsed solid-state lasers via laser diodes suffer from the drawback that the range of temperatures in which the laser diode can operate is rather limited, thereby also further limiting the possible range of operating frequencies/frequency settings of pulsed solid-state laser, especially for medical/surgical applications.

In addition current solutions to optimize the control of the temperature of laser diodes, i.e. for controlling the emission spectrum/emitted wavelength of the laser diodes, for the pumping of solid-state lasers are rather static, have a low latency for compensation of laser diode temperature changes and inter alia require complex and dedicated cooling mechanisms for the laser diodes, e.g. comprising thermo-electric coolers.

### PROBLEM

It is therefore the object of the present invention to provide improved means for pumping pulsed solid-state lasers. For example, it is in particular an object of the present invention to improve the efficiency and effectivity of diode-pumping a pulsed solid-state laser and to improve the stability and laser power output efficiency of a/the pulsed diode-pumped solid-state laser.

### Solution

According to the present invention, this object is achieved by a method and a laser system.

An exemplary method for operating a pulsed diode-pumped solid-state laser may comprise one, some or all of the following steps:

providing a pump light source for pumping a solid-state laser, wherein said pump light source comprises at least one laser diode unit configured for emitting a series of light pulses for pumping the solid-state laser,

modulating the series of light emission pulses of the at least one laser diode unit such that only the light pulses with a frequency close to or equal to a requested frequency setting of the solid-state laser are operated with a/the required pulse amplitude and/or a/the required pulse duration to trigger light emission of the solid-state laser,

and the at least one laser diode unit further being configured such that any other light pulses of the at least one laser diode unit are operated to not trigger light emission of the solid-state laser.

Herein, the terms required pulse amplitude and required pulse duration for triggering light emission of the solid-state laser can be inter alia understood as a required minimal pulse amplitude and as a required minimal pulse duration for triggering light emission of the solid-state laser.

Hence, in the modulation step exemplary described above, the light pulses with a frequency close to or equal to a requested frequency setting of the solid-state laser can be operated with pulse amplitudes that are equal to or larger than a/the required (minimal) pulse amplitude and/or with pulse durations that are equal to or larger than a/the required (minimal) pulse duration for triggering light emission of the solid-state laser.

Stated differently, the terms “required pulse amplitude” and “required pulse duration” can be understood as referring to a minimal pulse amplitude and/or a minimal pulse duration required to trigger light emission of the solid-state laser.

Furthermore, herein a frequency close to a requested frequency setting of the solid-state laser can inter alia be understood as a frequency within a predetermined interval around the requested frequency setting. For example, within an interval of +/−10% or within an interval of +/−20% of the requested frequency setting for the pulsed operation of the solid-state laser.

In other words, in the herein exemplary described method for operating a pulsed diode-pumped solid-state laser, only the by the at least one laser diode unit emitted light pulses with a frequency close to or equal to a requested frequency setting of the solid-state laser are operated with laser diode pumping parameters, such as, for example, pulse duration(s) and pulse amplitude(s), that are sufficient to trigger laser emission from the solid-state laser, i.e. only the by the at least one laser diode unit emitted light pulses with a frequency close to or equal to a requested frequency of the solid-state laser are provided with energies above the lasing threshold of the solid-state laser.

Any other light pulses emitted by the at least one laser diode unit, i.e. light pulses with frequencies not equal to a/the requested frequency of the solid-state laser and/or light pulses with frequencies outside, i.e. above or below, a/said predetermined interval around a/the requested frequency setting of the solid-state laser can be operated with laser diode pumping parameters, such as, for example, pulse duration(s) and pulse amplitude(s), that do not trigger a light emission of the solid-state laser.

Stated differently, light pulses emitted by the at least one laser diode unit with frequencies not equal to a/the requested frequency of the solid-state laser and/or light pulses with frequencies outside, i.e. above or below, a/said predetermined interval around a/the requested frequency setting of the solid-state laser can be operated/provided with energies below the lasing threshold of the solid-state laser.

Hence, between light pulses emitted by the solid-state laser, the at least one laser diode unit can continue injecting/pumping of the solid-state laser with light pulses, but at energies below the lasing threshold of the solid-state laser.

The herein exemplary described method steps for operating a pulsed diode-pumped solid-state laser and the herein exemplary described laser system and laser system configurations for operating a pulsed diode-pumped solid-state laser can inter alia significantly improve the laser power output of a solid-state laser operated in pulsed mode.

In-house experimental tests have surprisingly shown that significant increases of laser power output of a pulsed solid-state laser can be achieved. In particular, for example, increases in the maximum energy per laser pulse of up to 30% or more can be achieved as compared to known pulsed solid-state laser systems. This benefit is in particular notable in low frequency settings.

The reasons for this rather unexpected and surprisingly large beneficial technical effect can inter alia be understood from the fact that, due to the modulated pulsed operation of the laser diode unit comprising pulses with energies below, the lasing threshold of the solid-state laser can heat up the laser diode(s) of the laser diode unit more quickly, more effectively and more efficiently to reach a/the designated operation temperature of the laser diode(s) of the laser diode unit, thus enabling a faster shift of the wavelength of the light emitted by the laser diode(s) of the laser diode unit towards/to the designated operating wavelength of the laser diode(s) of the laser diode unit at which the gain medium/the laser crystal of the solid-state laser best absorbs the light emitted by the laser diode(s) of the laser diode unit.

Not only is the designated operating temperature of the laser diode(s) of the laser diode unit reached faster as compared to current systems, but the operating conditions of the laser diode(s), in particular its/their operating temperature, is kept more stable and accurate as compared to current systems. For example, since the laser diode(s) of the laser diode unit are not operated at full capacity, maximum energy or maximum current at all times during the pumping operation, also the risk of overheating the laser diode(s) of the laser diode unit and thereby again the risk of shifting the wavelength of the pump light emitted by the laser diode(s) of the laser diode unit to wavelengths which are absorbed less or not absorbed at all by the gain medium/the laser crystal of the solid-state laser and resulting in inefficient pumping and loss in laser power output of the solid-state laser is reduced.

Consequently, the pumping of the solid state laser is more stable, more efficient and more accurate, resulting also in a more stable laser output of the solid-state laser.

Thanks to the rapid reach of optimal operations of the laser diode(s) of the laser diode unit to rapidly establish optimal and efficient pumping of the solid-state laser, the methods and means described herein also enable fast changes of different operation modes of the solid-state laser, e.g. fast changes between different frequency settings of the solid-state laser, such as, for example, changing from high frequency settings/high repetition rate settings to low frequency settings/low repetition rate settings. Herein, high frequency settings/high repetition rate settings may, for example, refer to frequencies greater than 100 Hz or greater than 200 Hz, and low frequency settings/low repetition rate settings may refer to frequencies less than 100 Hz or less than 50 Hz.

The herein exemplary presented method steps for operating a pulsed diode-pumped solid-state laser and the herein exemplary described laser system configurations allow quasi-instantaneous changes between different frequency settings of the solid-state laser, for example, between low frequency settings and high frequency settings. In other words, the herein exemplary presented method steps for operating a pulsed diode-pumped solid-state laser and the herein exemplary described laser system configurations essentially eliminate the rather long time scales needed in conventional systems, which may require tens of seconds or minutes or even longer, for changes between different frequency settings of the solid-state laser.

Since the herein exemplary presented method steps for operating a pulsed diode-pumped solid-state laser and the herein exemplary described laser system configurations allow for more efficient and stable operations of the pulsed diode-pumped solid-state laser system, for example, a cooling mechanism for the laser diodes can be simplified and, for example, can be implemented by a water cooling mechanism. Consequently, more expensive and/or more cumbersome thermal regulation components for the laser diode(s) of the laser diode unit, such as thermo-electric coolers, can be dispensed with.

Hence, aside from providing a faster operation flow of the solid-state laser, improved power output of the solid-state laser, in particular in low frequency settings higher laser energies per laser pulse can be achieved as compared to known solid-state laser systems, and lower latencies for changing between different frequency settings of the solid-state laser, the herein exemplary described laser system configurations can further allow a simplified and more compact design of a diode-pumped solid-state laser system as compared to current systems.

The herein exemplary described means, laser system configurations and methods for operating a diode-pumped solid-state laser can be in particular suited for pulsed diode-pumped solid-state lasers for medical/surgical applications, e.g. for the treatment of biological tissue, and wherein the solid-state laser is being operated in pulsed mode in a frequency settings range of one to several hundreds of Hz.

For example, a selectable diode-pumped solid-state laser frequency setting can, for example, lie at a frequency at or below 300 Hz, in particular, for example, at or below 100 Hz.

For example, for the treatment of soft tissue with the herein described solid-state laser system a frequency setting with a frequency between 100-300 Hz or between 50-300 Hz may be applicable.

For the treatment or removal of harder components, such as urinary stones, for example, a frequency setting for the diode-pumped solid-state laser within the range of frequency settings from 3-100 Hz could be used.

Lower frequency settings for the diode-pumped solid-state laser together with higher energies/higher laser diode currents, e.g. with laser diode currents of up to 100 A or up to 200 A or higher, can be applied in order facilitate and speed up the fragmentations of stones that are to be removed from a patient. Such a treatment or operation mode can inter alia be referred to as fragmentation.

Smaller stone fragments can then subsequently be removed using higher frequency settings for the diode-pumped solid-state laser and lower energies/lower laser diode currents, e.g. laser diode currents of less than 100 A or less than 50 A. Such a treatment or operation mode can inter alia be referred to as dusting.

An exemplary method for operating a pulsed diode-pumped solid-state laser then may comprise the following setups and steps.

For example, the selectable/requestable/target laser frequency setting for an exemplary diode-pumped solid-state laser can be one of the following frequency settings: 5, 10, 20, 25, 50, 75 or 100 Hz and an exemplary laser diode unit can be designed/configured for emitting light pulses with a frequency of 100 Hz for a chosen pumping wavelength, e.g. a pumping wavelength optimized for a preferred/desired frequency setting of the solid-state laser, i.e. a desired operating mode of the solid-state laser.

For said exemplary frequency settings of the diode-pumped solid-state laser, the modulation of the light pulses emitted from/by the exemplary laser diode unit can be specified as follows.

For example, for a selected/requested solid-state laser frequency setting of 5 Hz, only every 20th light pulse of the laser diode unit can be operated with the required pulse amplitude and/or the required pulse duration to trigger light emission of the solid-state laser,

for a selected/requested solid-state laser frequency setting of 10 Hz only every 10th light pulse of the laser diode unit can be operated with the required pulse amplitude and/or the required pulse duration to trigger light emission of the solid-state laser,

for a selected/requested solid-state laser frequency setting of 20 Hz only every 5th light pulse of the laser diode unit can be operated with the required pulse amplitude and/or the required pulse duration to trigger light emission of the solid-state laser,

for a selected/requested solid-state laser frequency setting of 25 Hz only every 4th light pulse of the laser diode unit is operated with the required pulse amplitude and/or the required pulse duration to trigger light emission of the solid-state laser,

for a selected/requested solid-state laser frequency setting of 50 Hz only every 2nd light pulse of the laser diode unit is operated with the required pulse amplitude and/or the required pulse duration to trigger light emission of the solid-state laser,

and wherein for a selected/requested solid-state laser frequency setting of 75 Hz or 100 Hz all pulses of the laser diode unit are operated with the required pulse amplitude and/or the required pulse duration to trigger light emission of the solid-state laser.

Herein, an exemplary required pulse duration to trigger light emission of the solid-state laser can be a duration of up to 150 μs or can be a duration of up to 500 μs or more and a required exemplary pulse amplitude expressed in terms of electrical current applied to drive a laser diode/the laser diode unit can be up to 200 A or more and an exemplary electrical current applied to a laser diode/the laser diode unit that results in an emission of light pulse from the laser diode/the laser diode unit and that does not trigger a laser light emission from the solid-state laser can lie for example at 65 A or below.

It is inter alia further conceivable that the required pulse amplitude and/or the required pulse duration to trigger light emission of the solid-state laser are further modulated such that the values the required pulse amplitude(s) and/or the required pulse duration(s) of the pulses for triggering light emission of the solid-state laser can differ for a selected/requested solid-state laser frequency setting.

For example, in the exemplary cases listed above, it is conceivable that for a selected/requested solid-state laser frequency setting of 75 Hz or 100 Hz all pulses of the laser diode unit are operated with at least the required pulse amplitude and/or with at least the required pulse duration to trigger light emission of the solid-state laser, but that the pulses are not all having the same pulse amplitude and/or the same pulse duration. In other words in this example, the pulses may have at least an amplitude and a pulse duration required for triggering light emission of the solid-state laser but some or each may have different pulse amplitudes and/or different pulse durations.

It is further conceivable, that in general during operation of the pulsed diode-pumped solid-state laser, the electrical current provided to drive the at least one laser diode/laser diode unit can be non-zero at all times. This can inter alia further facilitate control of the operating temperature of the laser diode unit and, in particular, can further shorten the warm-up time of the laser diode unit and can therefore further optimize (i.e. reduce) latency of the laser system when operating the solid-state laser with/changing between different frequency settings.

An exemplary diode-pumped solid-state laser system that can be operated as exemplary described herein may comprise a solid-state laser and at least one laser diode unit that can be configured to operate according to any of the herein described exemplary described steps.

For example, the solid-state laser can be a Tm:YAG (Thulium:Ytterium Aluminium Garnate) solid-state laser.

An exemplary laser diode unit, an exemplary laser diode of the laser diode unit, may be configured for emitting light pulses with a wavelength of 780 nm or in the range of 778-782 nm at 100 Hz and with pulse durations of up to 500 μs and with amplitudes of up to a maximum current of 200 A or 250 A and at an exemplary temperature of 25° C.

An exemplary laser diode unit may comprise a single laser diode or a one-dimensional array of laser diodes, e.g. a diode-laser bar, or a two-dimensional array of laser diodes, e.g. a stack of diode-laser bars, or a three-dimensional array of laser diodes, e.g. comprising multiple stacks of diode-laser bars.

The herein described exemplary laser diode unit/the exemplary laser diode(s) of the exemplary laser diode unit can in particular be configured such that it can emit light with an emission spectrum substantially matching the absorption spectrum of the gain medium of the solid-state laser, when the laser diode unit/the laser diode(s) is/are operated at or close to the maximum of a predetermined electrical current supplied to the laser diode unit/the laser diode(s).

In particular, the herein described exemplary laser diode unit/the exemplary laser diode(s) of the exemplary laser diode unit can be configured/designed such as to emit light at/with a wavelength matching the wavelength at which the gain medium of the solid-state laser best absorbs light when the laser diode unit/the exemplary laser diode(s) is/are operated at/with a predetermined maximum electrical current and with/at a frequency (a/the operating frequency) which is the most critical for the intended use, i.e. the requested frequency setting f of the solid-state laser in terms of maximum laser power output/laser energy output E, such that the product E x f can be optimized.

Furthermore, the solid-state laser can be operated at/with a requested frequency setting as close as possible (e.g. within less than 10 or 20%) to a/the designed operating frequency of an exemplary laser diode unit/exemplary laser diode(s) to optimize the laser power output/laser energy output of the solid-state laser. Herein, the operating frequency of the exemplary laser diode unit/the exemplary laser diode(s) can be inter alia understood as pumping frequency of providing/emitting light pulses from the exemplary laser diode unit/the exemplary laser diode(s) to the solid-state laser that do not trigger a laser emission of the solid-state laser.

### BRIEF DESCRIPTION OF THE FIGURES

The following figures illustrate exemplary:

FIG. 1: Exemplary modulation of a series of light emission pulses of a laser diode unit

FIG. 2: Exemplary solid-state laser power/energy output at different frequency settings

### DETAILED DESCRIPTION

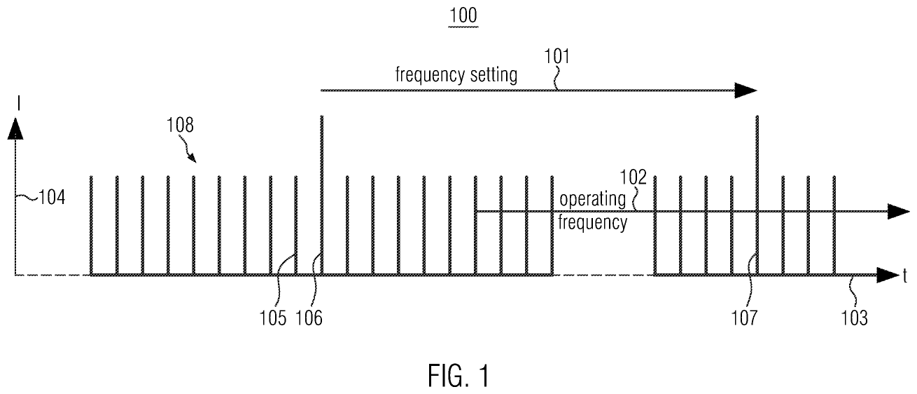

FIG. 1 illustrates an exemplary modulation 100 of a series 108 of light emission pulses 105, 106, 107 of an exemplary laser diode unit/laser diode(s) (not shown), with the ordinate axis 104 exemplary representing the amplitude or energy of the light pulses 105, 106, 107 or exemplary representing the electrical current(s) provided to the laser diode unit/laser diode that triggers/trigger said light pulses 105, 106, 107, and with the abscissa axis 103 exemplary representing time.

The exemplary pulses denoted with reference numerals 106 and 107 exemplary represent pulses emitted by the laser diode unit/laser diode(s) with an amplitude/energy that are required or sufficient to trigger light emission of a/the solid-state laser (not shown) that is receiving said pulses from the laser diode unit/laser diode(s), i.e. said pulses 106, 107 are operated at or above the lasing threshold of the solid-state laser and with a frequency 101 corresponding to a/the requested frequency setting of the pulsed solid-state laser.

Furthermore, the reference numeral 102 exemplary denotes an/the operating frequency of the exemplary laser diode unit/laser diode(s) with which the laser diode unit/laser diode(s) emits light pulses 105, 106, 107 to pump the solid-state laser and wherein the light pulses 105 with a frequency different from a/the requested frequency setting 101 are operated to not trigger light emission of the solid-state laser, i.e. are operated with an amplitude/energy below the lasing threshold of the solid-state laser.

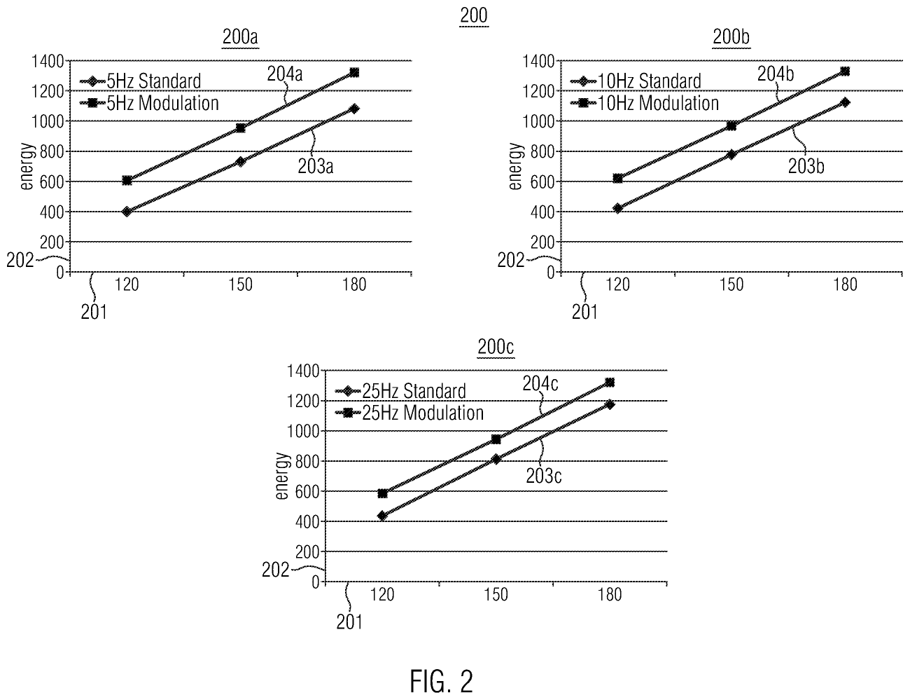

FIG. 2 exemplary illustrates test results 200 comparing the solid-state laser power/energy output at different requested frequency settings 200a, 200b, 200c comprising the requested frequency settings of 5 Hz, 10 Hz and 25 Hz when using standard pumping methods 203a, 203b, 203c as compared to pumping methods of a solid-state laser with a laser diode unit/laser diode(s) according to the herein described means and methods.

For example, the panel 200a shows the solid-state laser power/energy output 202 at a requested solid-state laser frequency setting of 5 Hz in dependence of the electrical current 201 supplied to a/the laser diode unit/laser diode(s) for a standard pumping 203a (line connecting the diamond-shaped measurement points) and for a modulated pumping 204a according to the method exemplary described herein (line connecting the square-shaped measurement points).

The panel 200b exemplary shows the solid-state laser power/energy output 202 at a requested solid-state laser frequency setting of 10 Hz in dependence of the electrical current 201 supplied to a/the laser diode unit/laser diode(s) for a standard pumping 203b (line connecting the diamond-shaped measurement points) and for a modulated pumping 204b according to the method exemplary described herein (line connecting the square-shaped measurement points).

The panel 200c exemplary shows the solid-state laser power/energy output 202 at a requested solid-state laser frequency setting of 25 Hz in dependence of the electrical current 201 supplied to a/the laser diode unit/laser diode(s) for a standard pumping 203c (line connecting the diamond-shaped measurement points) and for a modulated pumping 204c according to the method exemplary described herein (line connecting the square-shaped measurement points).

As can be seen when comparing the three different panels 200a, 200b, 200c which have the same scales, the gains/improvements in solid-state laser power/energy output efficiencies are significant for all requested frequency settings but highest for lower requested frequency settings.

For completeness, it is noted that for the shown results the laser diode unit/laser diode(s) for exemplary pumping the solid-state laser according to the method exemplary described herein, the exemplary operating frequency of the laser diode unit/laser diode(s) was 100 Hz.

However, the significant gains in laser power/energy output of a/the solid-state laser can be achieved also with other operating frequencies of the laser diode unit/laser diode(s).

Followed by two sheets comprising FIG. 1 and FIG. 2, wherein the reference numerals identify the following components.
100 exemplary modulation of light pulses for pumping a solid-state laser
101 exemplary (requested) frequency/frequency setting of a/the solid-state laser
102 exemplary operating frequency of exemplary laser diode unit/exemplary laser diode(s)
103 exemplary abscissa axis, exemplary time axis, t
104 exemplary ordinate axis, exemplary representing amplitude/energy of light pulses emitted by exemplary laser diode unit/exemplary laser diode(s) or exemplary electrical current, I, supplied to the laser diode unit/laser diode(s)
105 exemplary light pulse(s) emitted by exemplary laser diode unit/exemplary laser diode(s)
   that are operated with amplitudes/energies that do not trigger light emission by the solid-
   state laser, i.e. exemplary laser diode unit/exemplary laser diode pulses for pumping
   the solid-state laser that are operated below the lasing threshold of the solid-state laser
106, 107 exemplary light pulse(s) emitted by exemplary laser diode unit/exemplary laser diode(s) that are operated with sufficient high amplitudes/energies that trigger light emission by the solid-state laser, i.e. exemplary laser diode unit/exemplary laser diode pulses for pumping the solid-state laser that are operated at or above the lasing threshold of the solid-state laser
108 exemplary series of light pulses emitted by exemplary laser diode unit/laser diode(s) for pumping a/the solid-state laser
200 exemplary test results for achieved gains in laser power output of a/the solid-state laser in pulsed mode when pumped according to the herein described method as compared to standard pumping methods
200a, 200b, 200c exemplary test results for requested frequency settings 5 Hz, 10 Hz and 25 Hz of solid-state laser
201 exemplary abscissa axis, exemplary electrical current supplied to exemplary laser diode unit/exemplary laser diode(s), for example in units of Ampere
202 exemplary ordinate axis, exemplary laser power/energy output of exemplary solid-state laser in pulsed mode, for example specified in millijoule (mJ)
203a exemplary laser power/energy output of solid-state laser for standard pumping and a requested frequency setting of 5 Hz for the solid-state laser
204a exemplary laser power/energy output of solid-state laser for pumping according to the herein described method and a requested frequency setting of 5 Hz for the solid-state laser
203b exemplary laser power/energy output of solid-state laser for standard pumping and a requested frequency setting of 10 Hz for the solid-state laser
204b exemplary laser power/energy output of solid-state laser for pumping according to the herein described method and a requested frequency setting of 10 Hz for the solid-state laser
203c exemplary laser power/energy output of solid-state laser for standard pumping and a requested frequency setting of 25 Hz for the solid-state laser
204c exemplary laser power/energy output of solid-state laser for pumping according to the herein described method and a requested frequency setting of 25 Hz for the solid-state laser

1. A method for operating a pulsed diode-pumped solid-state laser comprising:
providing a pump light source for pumping a solid-state laser, said pump light source comprising at least one laser diode unit configured for emitting a series of light pulses for pumping the solid-state laser,
modulating the series of light emission pulses of the at least one laser diode unit such that only the light pulses with a frequency close to or equal to a requested frequency setting of the solid-state laser are operated with a required pulse amplitude and/or a required pulse duration to trigger light emission of the solid-state laser,
and such that any other light pulses emitted by the at least one laser diode unit are operated to not trigger light emission of the solid-state laser.

2. The method according to claim 1, wherein the requested frequency setting for the solid-state laser lies at a frequency below 300 Hz.

3. The method according to claim 1, wherein the requested frequency setting for the solid-state laser is one of the following frequency settings: 5, 10, 20, 25, 50, 75 or 100 Hz,
and wherein the at least one laser diode unit is operated to emit light pulses with a frequency of 100 Hz, and wherein for a requested solid-state laser frequency setting of 5 Hz, only every 20^{th} light pulse of the laser diode unit is operated with the required pulse amplitude and/or the required pulse duration to trigger light emission of the solid-state laser,
and wherein for a requested solid-state laser frequency setting of 10 Hz only every 10^{th} light pulse of the laser diode unit is operated with the required pulse amplitude and/or the required pulse duration to trigger light emission of the solid-state laser,
and wherein for a requested solid-state laser frequency setting of 20 Hz only every 5^{th} light pulse of the laser diode unit is operated with the required pulse amplitude and/or the required pulse duration to trigger light emission of the solid-state laser,
and wherein for a requested solid-state laser frequency setting of 25 Hz only every 4^{th} light pulse of the laser diode unit is operated with the required pulse amplitude and/or the required pulse duration to trigger light emission of the solid-state laser,
and wherein for a requested solid-state laser frequency setting of 50 Hz only every 2^{nd} light pulse of the laser diode unit is operated with the required pulse amplitude and/or the required pulse duration to trigger light emission of the solid-state laser,
and wherein for a requested solid-state laser frequency setting of 75 Hz or 100 Hz all pulses of the laser diode unit are operated with the required pulse amplitude and/or the required pulse duration to trigger light emission of the solid-state laser.

4. The method according to claim 1, wherein during operation of the pulsed diode-pumped solid-state laser, an electrical current provided to drive the at least one laser diode unit is non-zero at all times.

5. A diode-pumped solid-state laser system comprising:
a solid-state laser,
at least one laser diode unit configured to operate according to claim 1.

6. The diode-pumped solid-state laser system according to claim 5, wherein
the solid-state laser is a Tm:YAG laser.

7. The diode-pumped solid-state laser according to claim 5, wherein the at least one laser diode unit is configured to emit light pulses with a wavelength between 778 to 782 nm at 100 Hz and with pulse durations of up to 500 μs and with amplitudes of up to a maximum current of 250 A.

8. The diode-pumped solid-state laser according to claim 5, wherein the at least one laser diode unit comprises a single laser diode or a one-dimensional array of laser diodes or a two-dimensional array of laser diodes or a three-dimensional array of laser diodes.

9. The diode-pumped solid-state laser according to claim 8, wherein the at least one laser diode unit comprises the one-dimensional array of laser diodes, and wherein the one-dimensional array of laser diodes is a diode laser bar.

10. The diode-pumped solid-state laser according to claim 8, wherein the at least one laser diode unit comprises the two-dimensional array of laser diodes, and wherein the two-dimensional array of laser diodes is a stack of diode laser bars.

11. The diode-pumped solid-state laser according to claim 8, wherein the at least one laser diode unit comprises the three-dimensional array of laser diodes, and wherein the three-dimensional array of laser diodes is multiple stacks of diode laser bars.
